(19) United States
(12) Patent Application Publication
(10) Pub. No.: US 2024/0257556 A1
(43) Pub. Date: Aug. 1, 2024

(54) MEDIA CONTENT METRICS SYSTEMS UTILIZING FACIAL EXPRESSION DATA

(71) Applicant: David K. Pederson, Mulino, OR (US)

(72) Inventor: David K. Pederson, Mulino, OR (US)

(21) Appl. No.: 18/635,820

(22) Filed: Apr. 15, 2024

Related U.S. Application Data
(63) Continuation-in-part of application No. 18/587,630, filed on Feb. 26, 2024.
Application No. 18/587,630 is a continuation-in-part of application No. 18/082,866, filed on Dec. 16, 2022, now Pat. No. 11,989,974.
Application No. 18/082,866 is a continuation of application No. 16/859,727, filed on Apr. 27, 2020, now Pat. No. 11,568,680.

(51) Int. Cl. G06V 40/16; A61B 5/00; A61B 5/021; A61B 5/16

(52) U.S. Cl. CPC G06V 40/174; A61B 5/0022; A61B 5/0077; A61B 5/021; A61B 5/165; A61B 5/4848; A61B 5/681; A61B 5/6898; G06V 40/166; G06V 40/172

(57) ABSTRACT

Media content metrics systems including a facial expression detection device, a system processor, and a media device. The system processor is in data communication with the facial expression detection device and is configured to execute stored computer executable system instructions. The media device is controllably coupled to the system processor and configured to play and stop playing a media file in response to playback instructions from the system processor. The computer executable system instructions include presenting media content, receiving facial expression parameter data, receiving current facial expression data, comparing the current facial expression data to the facial expression parameter data, identifying whether the current facial expression data satisfies target facial expression criteria, and establishing media metrics data for the media content based on the comparison of the current facial expression data to the target facial expression criteria.

### PRIORITY CLAIM

This application claims priority to copending U.S. patent application Ser. No. 18/082,866, filed on Dec. 16, 2022, and Ser. No. 18/587,630 filed on Feb. 26, 2024, and U.S. patent Ser. No. 11/568,680, issued Jan. 31, 2023; which are hereby incorporated by reference in their entirety for all purposes.

### BACKGROUND

Smiling is known to non-verbally communicate emotions, like happiness and joy, and other messages, such as approval between people. Lesser known is the fact that smiling can provide health benefits, both to the person observing another smiling and to the person smiling. This document will focus on the therapeutic health benefits from smiling to the person who is smiling.

Genuine smiling, often called a Duchenne smile, is a particular manner of smiling that has distinct health benefits. A genuine smile improves mood, reduces blood pressure, reduces stress, reduces pain, strengthens the immune system, strengthens relationships, increases attractiveness, and improves longevity. A genuine smile is characterized by activating muscles near the eyes and cheeks in contrast to a fake or perfunctory smile that merely involves shaping the lips.

Conventional facial recognition systems are capable of detecting certain expressions on a person's face, but are not designed to detect genuine smiles. Further, existing facial recognition systems do not include features to train people how to execute a genuine smile. Conventional facial recognition systems also lack features to encourage people to execute a genuine smile with a given frequency, for a given amount of time, and/or in response to a physiological trigger.

Thus, there exists a need for smile detection systems that improve upon and advance the design of known facial recognition systems. Examples of new and useful smile detection systems relevant to the needs existing in the field are discussed below.

### SUMMARY

The present disclosure is directed to media content metrics systems including a facial expression detection device, a system processor, and a media device. The system processor is in data communication with the facial expression detection device and is configured to execute stored computer executable system instructions. The media device is controllably coupled to the system processor and configured to play and stop playing a media file in response to playback instructions from the system processor.

The computer executable system instructions include presenting media content, receiving facial expression parameter data, receiving current facial expression data, comparing the current facial expression data to the facial expression parameter data, identifying whether the current facial expression data satisfies target facial expression criteria, and establishing media metrics data for the media content based on the comparison of the current facial expression data to the target facial expression criteria.

### BRIEF DESCRIPTION OF THE DRAWINGS

FIG. 1 is a perspective view of a system for detecting when a person exhibits a smile with therapeutic benefits incorporated into a watch on a person's wrist.

FIG. 2 is a schematic view of the system shown in FIG. 1.

FIG. 3 is a flow diagram of computer executable instruction steps that the system shown in FIG. 1 is programmed to follow.

FIG. 4 is a flow diagram showing additional computer executable instruction steps associated with the step of prompting the person to exhibit a facial expression.

FIG. 5 is a flow diagram showing alternative additional computer executable instruction steps associated with the step of prompting the person to exhibit a facial expression.

FIG. 6 is front view of a second example of a system for detecting when a person exhibits a smile with therapeutic benefits, the system incorporated into a smart phone.

FIG. 7 is a perspective view of a third example of a system for detecting when a person exhibits a smile with therapeutic benefits incorporated into a computer with a camera.

FIG. 8 is a is a schematic view of a media content metrics system.

FIG. 9 is a flow diagram of computer executable instructions utilized by the system shown in FIG. 8.

FIG. 10 is a flow diagram of alternative computer executable instructions that may be utilized by a media content metrics system.

FIG. 11 is a flow diagram of alternative computer executable instructions that may be utilized by a media content metrics system.

### DETAILED DESCRIPTION

The disclosed media content metrics systems will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various media content metrics systems are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

### Therapeutic Smile Detection Systems

With reference to the figures, therapeutic smile detection systems will now be described. The systems discussed herein function to detect when a user is executing a genuine smile, also known as a Duchenne smile. The systems described in this document also function to train a user to execute a genuine smile. Another function of the systems described herein is to encourage people to execute a genuine smile to promote associated therapeutic benefits and to help establish healthy habits.

The reader will appreciate from the figures and description below that the presently disclosed systems address many of the shortcomings of conventional smile detection systems. For example, the systems described herein are sophisticated enough to detect genuine smiles in contrast to conventional facial recognition systems, which can detect only certain general expressions on a person's face. Further, the presently disclosed systems train people how to execute a genuine smile to enable them to experience the health benefits of genuine smiles. The systems discussed in this document improve over conventional facial recognition systems by encouraging people to execute a genuine smile with a given frequency, for a given amount of time, and or in response to a physiological trigger.

### Contextual Details

Ancillary features relevant to the smile detections described herein will first be described to provide context and to aid the discussion of the smile detection systems.

### Person

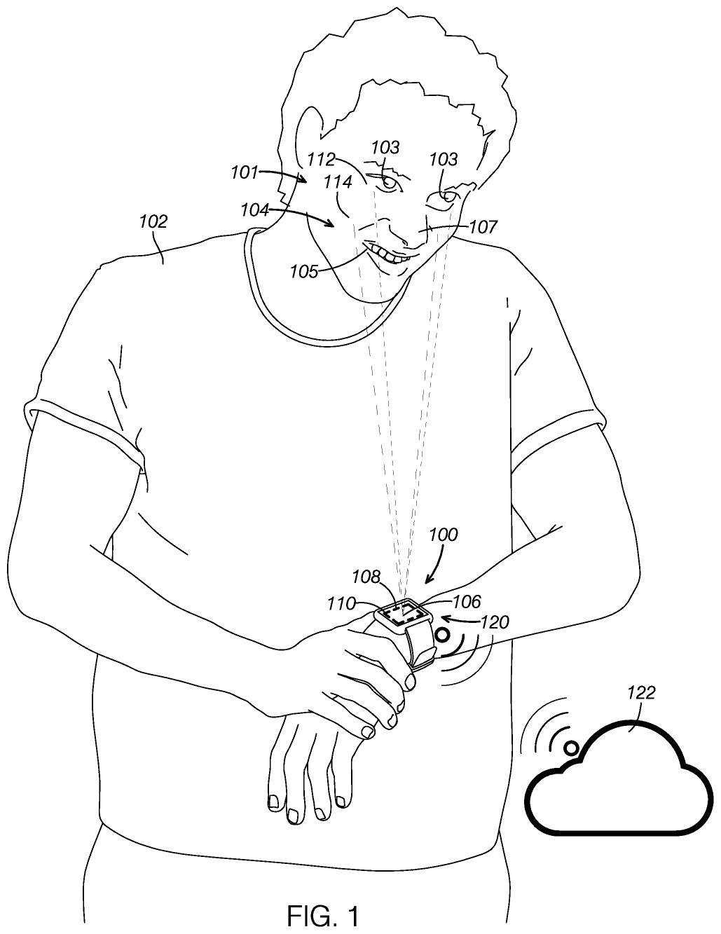

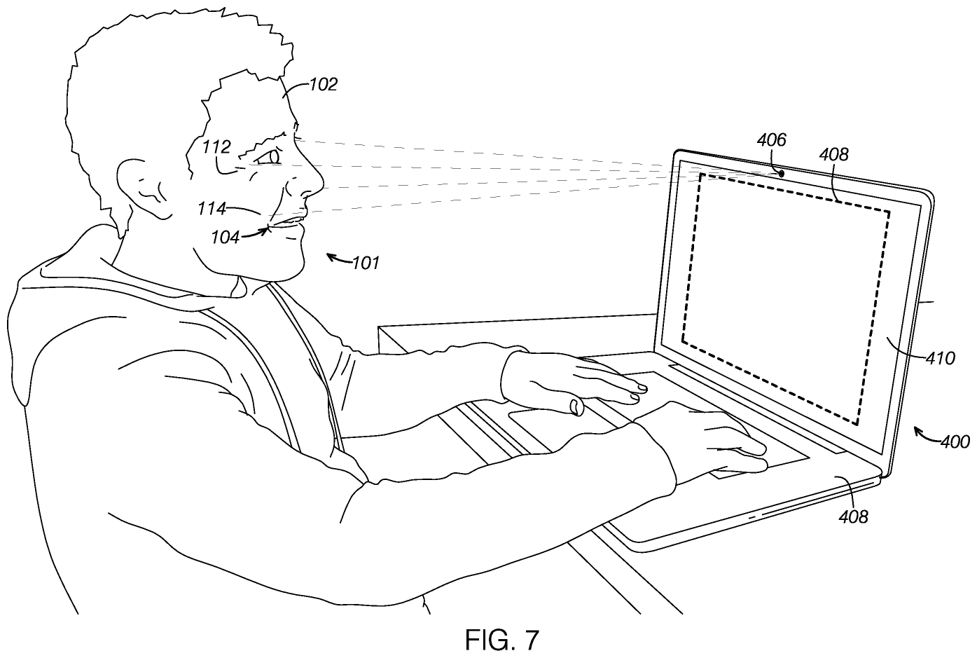

The smile detection systems described herein function to detect a smile and other facial expressions of a person, which may also be referred to as a user. With reference to FIGS. 1 and 7, a person 102 is depicted using smile detection system 100 in FIG. 1 and using smile detection system 400 in FIG. 7. Person 102 includes a face 101, eyes 103, a mouth 105, and a nose 107. Further, person 102 includes orbicularis oculi muscles 112 near eyes 103 and zygomatic major muscles 114 near mouth 105. As shown in FIGS. 1 and 7, person 102 may exhibit a smile 104.

### Smile Detection System Embodiment One

With reference to FIGS. 1-5, a first example of a smile detection system, smile detection system 100, will now be described. Smile detection system 100 includes a facial expression detection device 106, a system processor 108, and a display 110. In some examples, the smile detection system does not include one or more features included in smile detection system 100. For example, some smile detection system examples do not include a display. In other examples, the smile detection system includes additional or alternative features.

### Facial Expression Detection Device

Facial expression detection device 106 is configured to acquire facial expression data. The facial expression data may include information about the position of facial features, such as the eyes 103, nose 107, mouth 105, and ears of person 102. The facial expression data may be more granular, such as the position of specific facial muscles, such as the zygomatic major muscles 114 and/or the orbicularis oculi muscles 112 of person 102. The facial expression data may include information related to how facial features have moved by comparing the position of a given feature over time.

Additionally or alternatively to information about particular facial features, the facial expression data may include information about expressions the person is exhibiting. The expression a person is exhibiting may be determined by combining information about facial features and/or by associating expressions with defined indicators. For example, wrinkles near a person's eyes or upturned lips may be defined as indicators for a smile.

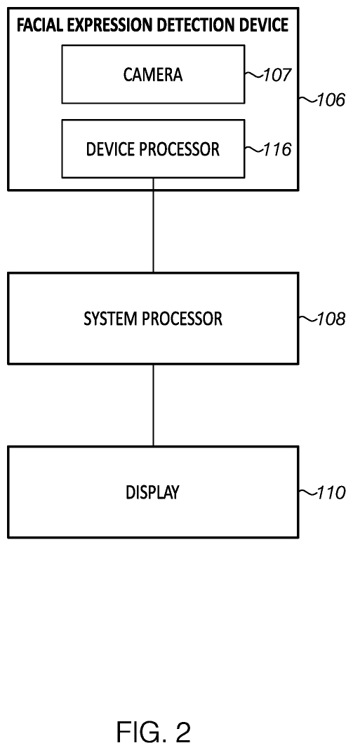

As can be seen in FIGS. 1 and 2, facial expression detection device 106 includes a camera 107 and a device processor 116. Camera 107 is configured to collect facial expression data from person 102.

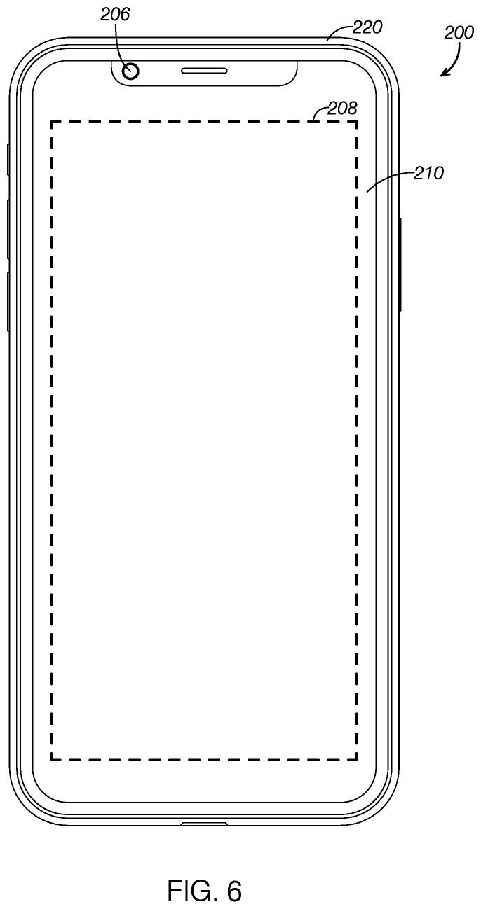

The camera may be any currently know or later developed camera suitable for collecting facial expression data from a person. In the example shown in FIG. 1, camera 107 is incorporated into a watch 120. In the example shown in FIG. 6, the camera is incorporated into a smartphone 220. In the example shown in FIG. 7, the camera is incorporated into a laptop computer 420.

Device processor 116 is configured to execute stored computer executable facial recognition instructions. The device processor may be any currently known or later developed processor suitable for executing computer executable instructions. The facial recognition instructions may be customized for detecting smiles with the facial expression detection device or may be more generally applicable facial recognition instructions.

As can be seen in FIG. 1, facial expression detection device 106 is incorporated into a watch 120. In particular, watch 120 is a smart watch with various computing features. However, the watch may be a traditional watch without computing features beyond the facial expression detection device. FIG. 6 depicts an example where a facial expression detection device 206 is incorporated into a handheld computing device in the form of a smartphone 220. FIG. 7 depicts an example where a facial expression detection device 406 is incorporated into a personal computing device in the form of a laptop computer 420.

### System Processor

System processor 108 is configured to execute stored computer executable system instructions 300. As shown in FIG. 2, system processor is in data communication with facial expression detection device 106 and with display 110.

The system processor may be any currently known or later developed processor suitable for executing computer executable instructions. The system instructions may include instructions customized for detecting a smile 104 with facial expression detection device 106, such as system instructions 300, and more generally applicable facial recognition instructions.

### Computer Executable System Instructions

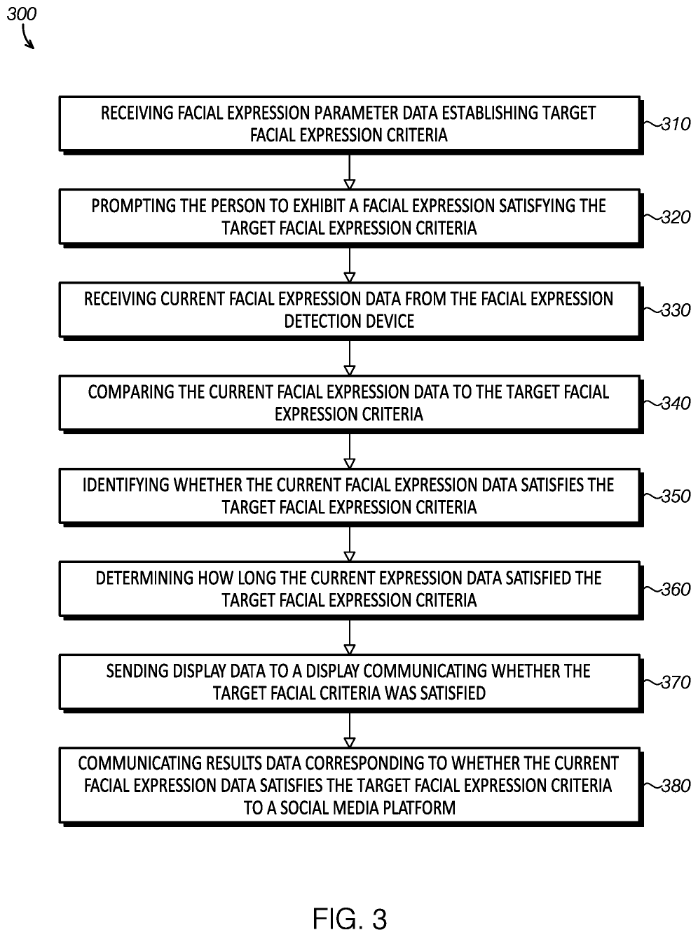

With reference to FIGS. 3-5, a particular set of computer executable system instructions, system instructions 300, will be described. The reader should appreciate that additional or alternative system instructions may be used in different examples.

As shown in FIG. 3, system instructions 300 include the step of receiving facial expression parameter data establishing target facial expression criteria at step 310. The target facial expression criteria define a smile with therapeutic benefits. Examples of smiles with therapeutic benefits include a genuine smile, which is also known as a Duchenne smile.

The target facial expression criteria may define a smile with therapeutic benefits as occurring when zygomatic major muscles 114 of person 102 contract to a selected extent. Additionally or alternatively, the target facial expression criteria may define a smile with therapeutic benefits as occurring when orbicularis oculi muscles 112 of person 102 contract to a selected extent.

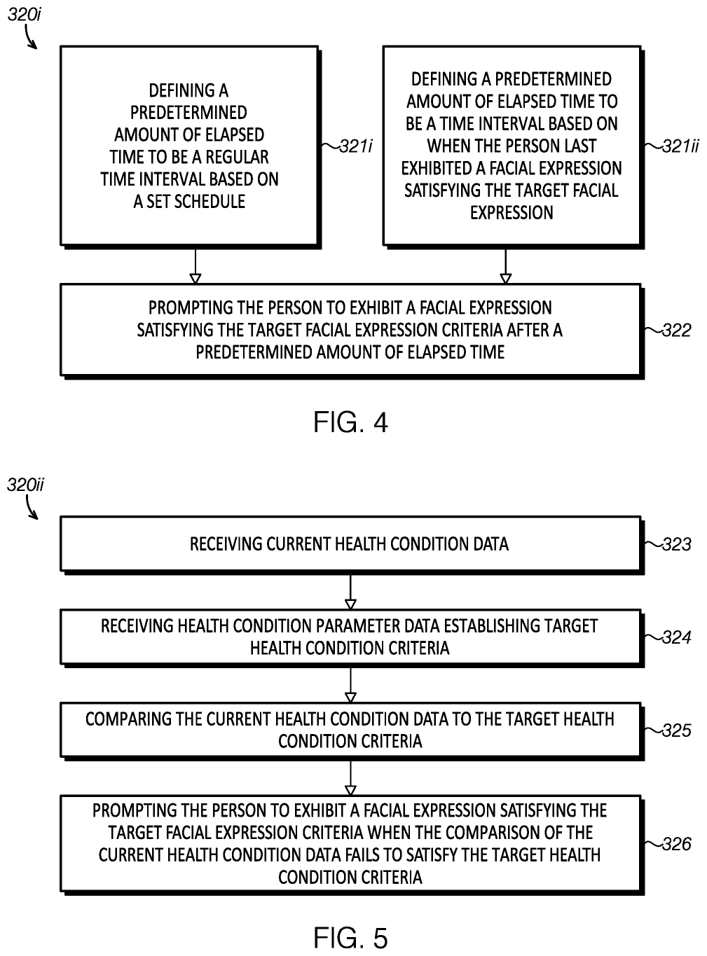

As can be seen in FIG. 3, system instructions 300 include prompting person 102 to exhibit a facial expression satisfying the target facial expression criteria at step 320. FIG. 4 depicts one example of prompting a person to exhibit a facial expression at step 320i. FIG. 5 depicts another example of prompting a person to exhibit a facial expression at step 320ii.

Prompting a person to exhibit a facial expression at step 320 may occur after a predetermined amount of elapsed time, such as shown at steps 321i and 321ii of step variation 320i in FIG. 4. Additionally or alternatively, prompting a person to exhibit a facial expression at step 320 may occur when a comparison of current health condition data fails to satisfy target health condition criteria, such as shown at step 326 of step variation 320ii in FIG. 5.

With reference to FIG. 4, the reader can see additional steps involved with prompting person 102 to exhibit a facial expression satisfying the target facial expression criteria in step variation 320i. Step 321i includes defining a predetermined amount of elapsed time to be a regular time interval based on a set schedule. Additionally or alternatively to step 321i, step 321ii includes defining a predetermined amount of elapsed time to be a time interval based on when person 102 last exhibited a facial expression satisfying the target facial criteria. Step variation 320i includes prompting person 102 to exhibit a facial expression satisfying the target facial expression criteria after a predetermined amount of elapsed time at step 322.

With reference to FIG. 5, the reader can see that prompting person 102 to exhibit a facial expression satisfying the target facial expression criteria in step variation 320ii includes receiving current health condition data at step 323. In the step 320ii example, the current health condition data corresponds to the health of person. The current health condition data may include blood pressure data, body temperature data, pulse rate data, metabolic data, and various other types of physiological data. In certain examples, the current health condition data includes the current facial expression data, such as whether person 102 is smiling, scowling, frowning, or tensing facial muscles.

At step 324, prompting a person to exhibit a facial expression at step 320ii further includes receiving health condition parameter data establishing target health condition criteria. The target health condition criteria may define conditions corresponding to low stress or other healthy states of being. The target health condition criteria may include defined ranges for blood pressure, body temperature, pulse rate, metabolic rates, and various other types of physiological parameters. The defined ranges may be selected to correspond to ranges known to promote healthy lifestyles.

In the example shown in FIG. 5, prompting a person to exhibit a facial expression at step 320ii also includes comparing the current health condition data to the target health condition criteria of the health condition parameter data at step 325. The comparison performed at step 325 may be used to track a user's health metrics. Additionally or alternatively, the comparison performed at step 325 may be used to trigger prompts to exhibit a facial expression.

For example, step 326 includes prompting person 102 to exhibit a facial expression satisfying the target facial expression criteria when the comparison of the current health condition data fails to satisfy the target health condition criteria. In some examples, the user is prompted to exhibit a desired facial expression immediately when the current health condition data fails to satisfy the target health condition criteria. In some examples, the user is prompted to exhibit a desired facial expression when the current health condition data fails to satisfy the target health condition criteria for a predetermined amount of time.

Returning focus to FIG. 3, system instructions 300 include receiving current facial expression data from facial expression detection device 106 at step 330. The current facial expression data may be received via a wired or wireless data connection.

At step 340, system instructions 300 include comparing the current facial expression data to the target facial expression criteria of the facial expression parameter data. After comparing the facial expression data to the target facial expression criteria at step 340, system instructions 300 include identifying whether the current facial expression data satisfies the target facial expression criteria at step 350.

In the present example, system instructions 300 include optional step 360 of determining how long the current expression data satisfied the target facial expression criteria. Other examples do not include tracking how long the target facial expression criteria was satisfied. In applications where maintaining a facial expression for a prescribed length of time is desired, such as maintaining a genuine smile for a given length of time to provide desired health benefits, tracking how long the target facial expression criteria was satisfied can assist with encouraging a user to maintain the facial expression for the prescribed time. Tracking how long the target facial expression criteria was satisfied can also help with communicating or reporting facial expression performance results.

At step 370, system instructions 300 include sending display data to display 110. The display data may communicate whether, how often, and/or for how long the target facial criteria was satisfied. The display data may also include health information, such as health benefits resulting from exhibiting facial expressions satisfying the target facial criteria.

Sending the display data to display 110 may assist the user to understand his or her facial expression performance and to make adjustments accordingly. In some examples, sending the display data to a display is performed as part of a game or contest where a user is encouraged to exhibit a desired facial expression. For example, a user may be assigned points, be awarded virtual prizes, or progress to new places in a virtual world when meeting facial expression parameters communicated to the user in the form of a game or entertainment experience.

At step 380 in FIG. 3, the reader can see that system instructions 300 include communicating results data to a social media platform, such as social media platform 122 depicted conceptually in FIG. 1. The results data may correspond to whether the current facial expression data satisfies the target facial expression criteria. Additionally or alternatively, the results data may include how often, and/or for how long the target facial criteria was satisfied. In some examples, the results data may also include health information, such as health benefits resulting from exhibiting facial expressions satisfying the target facial criteria. In examples where the system instructions incorporate or work in conjunction with a game, the results data may include the points, virtual prizes, or progress the user has achieved in the game that utilizes facial expressions.

### Display

Display 110 functions to display display data to person 102. As shown in FIG. 2, display 110 is in data communication with system processor 108. Display 110 and system processor 108 may communicate data via a wired or wireless connection.

The display may be any currently known or later developed type of display for displaying data. In the example shown in FIG. 3, display 110 is a watch screen. In the example shown in FIG. 6, display 210 is a smartphone screen. In the example shown in FIG. 7, display 410 is a laptop computer screen.

### Additional Embodiments

The discussion will now focus on additional smile detection system embodiments. The additional embodiments include many similar or identical features to smile detection system 100. Thus, for the sake of brevity, each feature of the additional embodiments below will not be redundantly explained. Rather, key distinctions between the additional embodiments and smile detection system 100 will be described in detail and the reader should reference the discussion above for features substantially similar between the different smile detection system examples.

### Second Embodiment

Turning attention to FIG. 6, a second example of a smile detection system, smile detection system 200, will now be described. As can be seen in FIG. 6, smile detection system 200 includes a facial expression detection device 206, a system processor 208, and a display 210. A distinction between smile detection system 200 and smile detection system 100 is that smile detection system 200 is incorporated into a smart phone 220 rather than into watch 120.

### Third Embodiment

Turning attention to FIG. 7, a third example of a smile detection system, smile detection system 400, will now be described. As can be seen in FIG. 7, smile detection system 400 includes a facial expression detection device 406, a system processor 408, and a display 410. A distinction between smile detection system 400 and smile detection system 100 is that smile detection system 400 is incorporated into a laptop computer 420 rather than into watch 120.

### Fourth Embodiment

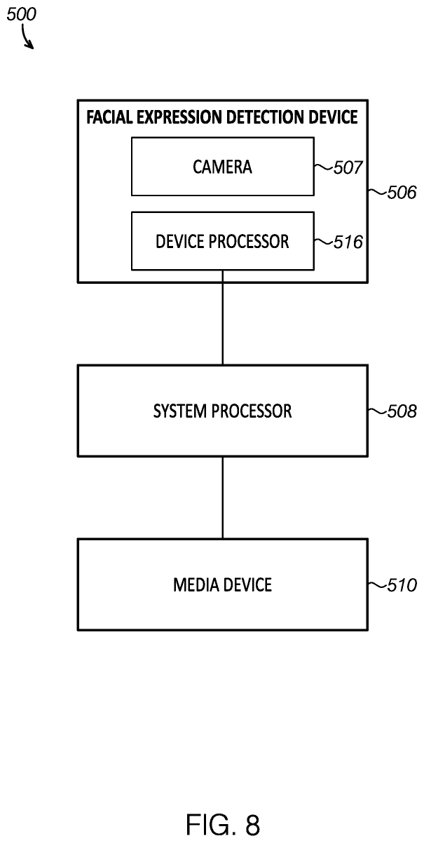

Turning attention to FIGS. 8-11, media content metrics systems based on a person exhibiting a smile with therapeutic benefits will now be described. As can be seen in FIG. 8, media content metrics system 500 includes a facial expression detection device 506, a system processor 508, and a media device 510. The components of media playback system 500 and the computer executable instructions executed by media playback system 500 are described in more detail below.

Facial expression detection device 506 is configured similarly to facial expression detection devices 106, 206, and 406 described above. As with the devices described above, facial expression detection device 506 is configured to acquire facial expression data.

As shown in FIG. 8, facial expression detection device 506 includes a camera 507 and a device processor 516. As further shown in FIG. 8, facial expression detection device 506 is controllably coupled to system processor 508. Facial expression detection device 506 may be incorporated into a handheld computing device, a watch, a television, a laptop computer, a desktop computer, and any suitable device.

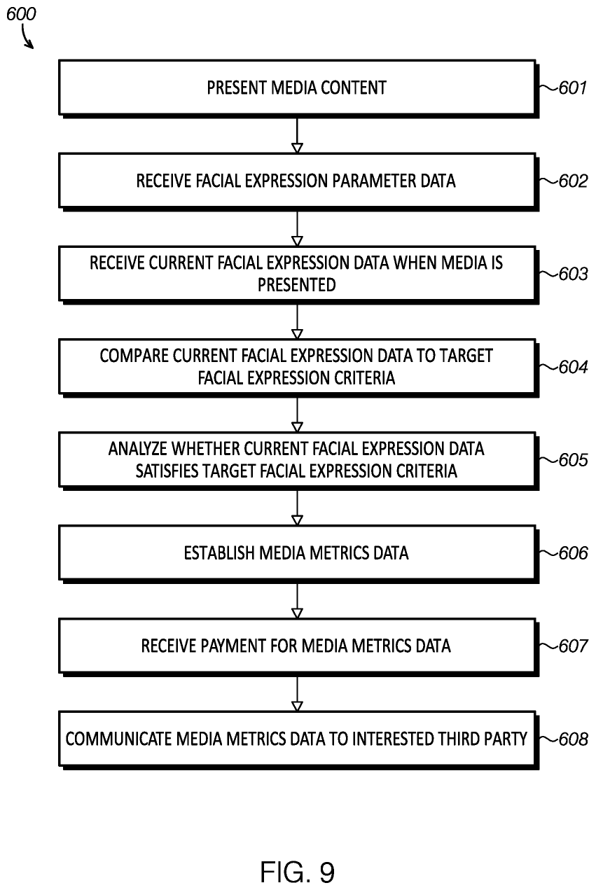

System processor 508 is configured similarly to system processors 108, 208, and 408 described above. As shown in FIG. 8, system processor 508 is controllably coupled to facial expression detection device 506 and media device 510. System processor 508 is configured to execute computer executable system instructions for establishing media metrics data and media playback instructions. Representative computer executable system instructions 600, 700, and 800 are shown in FIGS. 9-11 and described below.

Media device 510 is configured to play and stop playing media files in response to media playback instructions from system processor 508. In the examples discussed herein, media device 510 receives playback instructions to play and stop playing media files from system processor 508. As shown in FIG. 8, media device 510 is controllably coupled to system processor 508. Media device 510 can access media files on an external device, such as a media server, or store media files in internal memory.

### Computer Executable System Instructions

The computer executable system instructions function to establish media metrics data based on smile detection data. Further, the computer executable system instructions serve to enable system processor 508 to deliver media metrics data to interested parties. In some examples, subsequent media content is selected for presentation to a user based on whether the user exhibited a smile or other facial expression that indicated initially presented media content was well received.

Delivering media metrics data to interested parties may provide useful feedback to media content creators regarding how their media was received by content consumers. For example, the feedback may inform marketing content creators the extent to which their marketing content was effective. Alternatively, the media metrics data may inform entertainment media content creators and advertisers how popular entertainment media content is with viewers. The popularity of entertainment media content may be relevant to setting prices to run advertisements during presentations of the entertainment media content.

### Instructions Set 1

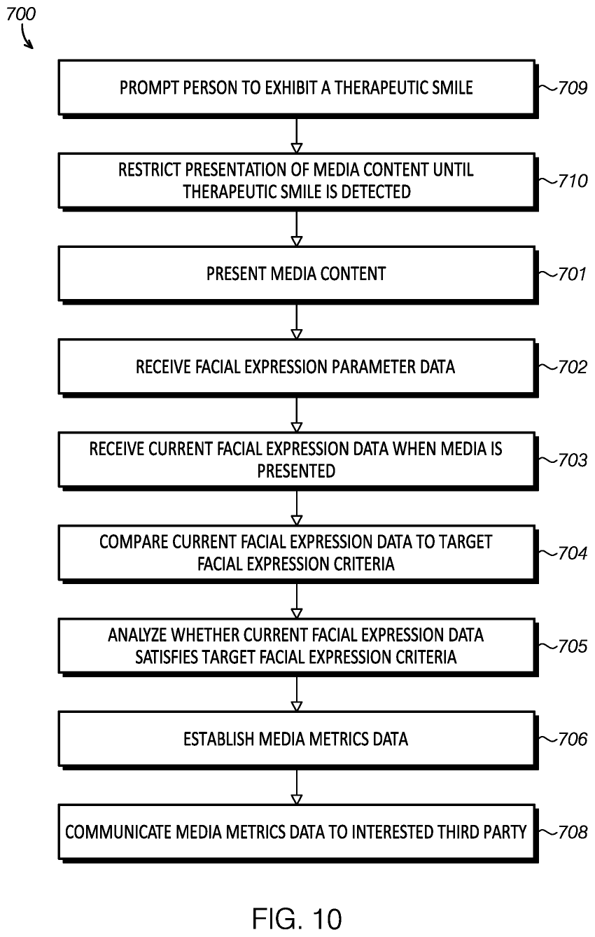

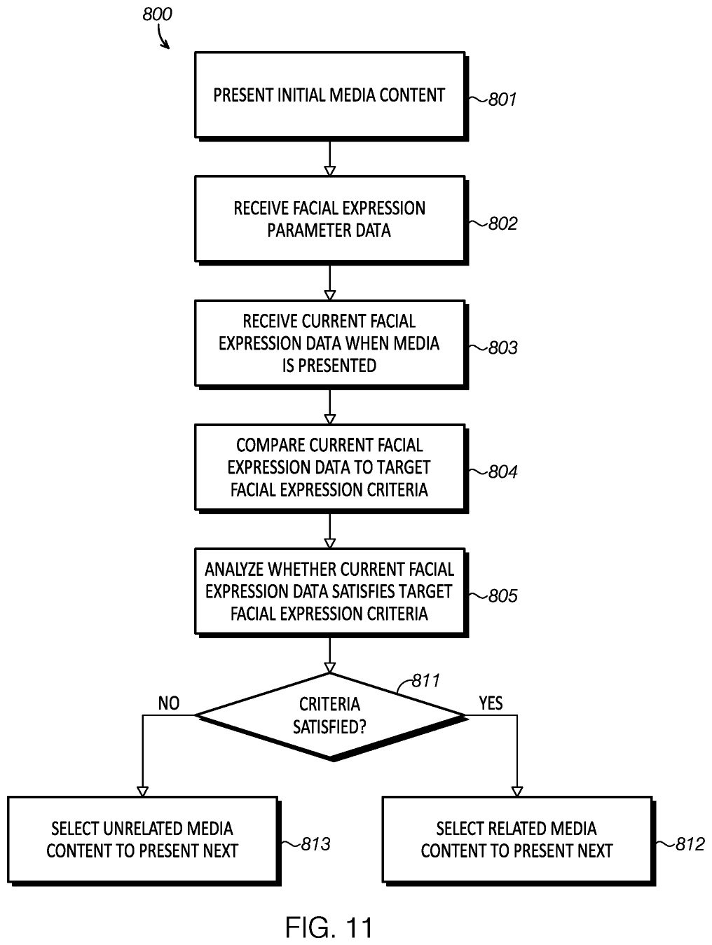

With reference to FIG. 9, a first set of computer executable system instructions, system instructions 600, will be described. Additional or alternative system instructions 700 and 800 are shown in FIGS. 10 and 11, respectively, and described below.

### Presenting Media Content

Presenting media content to a user at step 601 on media device 510 establishes the subject matter on which the media metrics data will be based. A wide variety of media may be presented, including media created for entertainment, educational, news, or advertisement purposes.

The media content presented at step 601 may be a video covering a wide variety of subject matter and forms. For example, the video may be a movie, television show, news presentation, or instructional video. In some examples, the media content delivered at step 601 is a short personal video by an amateur videographer. In other examples, the videos presented to the user are professionally produced.

In addition or alternatively to videos, the media content may be music, literary works, pictures, and the like. The media content may be any currently known or later developed type of media people may consume.

The media content may be an advertisement for a product or service or include an advertisement. In some examples, an advertisement is displayed during another video presented for entertainment, news, or educational purposes. Advertisement media content may be a video, an audio clip, a still picture, text, or any other form of media.

In some examples, such as depicted in FIG. 11, system processor 500 delivers media content to the user multiple times successively. The media content delivered one after another may be related, such as different episodes pf a television series, different songs from an album, or sequels or prequels of a movie series. In other examples, the media content items delivered one after another are unrelated.

### Receiving Facial Expression Parameter Data

Receiving facial expression parameter data at step 602 enables system 500 to evaluate whether the smile exhibited in step 601 meets criteria necessary for therapeutic benefits. The facial expression parameter data establishes target facial expression criteria defining a smile with therapeutic benefits.

The target facial expression criteria may include a variety of indicators known to be involved with therapeutic smiles or other facial expressions. For example, the target facial expression criteria may include the zygomatic major muscles of the person contracting to a selected extent. Additionally or alternatively, the target facial expression criteria may include the orbicularis oculi muscles of the person contracting to a selected extent.

### Receive Current Facial Expression Data

Receiving current facial expression data at step 603 enables system 500 to evaluate whether the user is exhibiting a therapeutic smile or other therapeutic facial expression. The current facial expression data received at step 603 corresponds to the facial expression of the person at a given time, such as when the user is prompted to exhibit a smile at step 601.

In system 500, the current facial expression data is obtained at step 603 from facial expression detection device 506. Camera 507 and device processor 516 of facial expression detection device 506 cooperate to acquire image data of a person's face, process the raw image data into current facial expression data, and to output the current facial expression data to system processor 508.

### Comparing and Analyzing Facial Expression Data

Comparing the current facial expression data to the target facial expression criteria at step 604 enables analysis of the current facial expression data in step 605. Analyzing the current facial expression data at step 605 enables system 500 to determine if the user has exhibited a therapeutic smile or other therapeutic facial expression. As shown in FIG. 9, system 500 uses the step 605 analysis to generate media metrics data at step 606.

At step 604, the current facial expression data is compared to the target facial expression criteria of the facial expression parameter data received in step 603. Any currently known or later developed means of comparing the facial expression data to the target facial expression criteria may be used.

As shown in FIG. 9, the comparison data obtained from step 604 is analyzed to determine if the user has exhibited a therapeutic smile or other therapeutic facial expression at step 605. A wide variety of analysis methodologies may be employed, such as statistical analysis or assigning a score to the comparison data and vetting the score against a predetermined score threshold.

In some examples, the computer executable system instructions cause system processor 508 to identify whether the current facial expression data satisfies the target facial expression criteria and to assign a value to a satisfaction variable. For example, system processor 508 may assign a satisfaction variable a binary value of true if the current facial expression data satisfies the target facial expression criteria. Conversely, system processor 508 may assign the satisfaction variable a binary value of false if the current facial expression data does not satisfy the target facial expression criteria.

### Establish Media Metrics Data

Media metrics data provides useful information for how a user of system 500 perceives media content presented at step 601. At a high level, the media metrics data reveals if a user liked media content or not. With more granularity, the media metrics data can indicate the extent to which a user liked or disliked media content, which portions of media content the user responded to most, and how long the media content captured the user's interest or attention.

The media metrics data may be useful to a variety of parties. For example, creators of the media content presented to the user may be interested in the media metrics data to assess how well the media content was received by the user. Further, manufacturers, distributors, or retailers of a product advertised in marketing media content may be interested in the media metrics data to evaluate how effectively the content marketed their product to a user of system 500.

At step 606, system processor 508 establishes media metrics data for the media content. The media metrics data established at step 606 is based on the comparison of the current facial expression data to the target facial expression criteria at steps 604 and 605.

The media metrics data may take a variety of forms. For example, the media metrics data may include whether the current facial expression data satisfies the target facial expression criteria, such as via a satisfaction variable with binary values of true or false. In addition or alternatively to binary satisfaction values, the media metrics data may include data indicating how long the current expression data satisfied the target facial expression criteria. Similarly, the media metrics data may include data indicating how quickly the current expression data failed to satisfy the target facial expression criteria.

At step 606, system processor 508 generates a satisfaction score by compiling selected aspects of the media metrics data. The satisfaction score corresponds to how well the current expression data satisfied the target facial expression criteria. The satisfaction score may be reflected as numerical scale, such as a value between 1 and 100, an alphabetic grade, such as a letter grade value between A and F, or a percentage value.

### Communicating Media Metrics Data to a Third Party

Communicating media metrics data to third parties informs third parties how media content was perceived by the user consuming the media presented to him or her. Optionally, communicating media metrics data to third parties enables monetizing the media metrics data by limiting the data to parties willing to pay for it.

Method 600 encompasses monetizing the media metrics data by requiring payment for the media metrics data. In particular, as shown in FIG. 9, optional step 607 is waiting to communicate the media metrics data to a third party until a payment record indicates that the third party has paid a predetermined fee to receive the media metrics data. The third party may pay for the satisfaction score and/or other metrics data in advance or on demand when the media metrics data is available to communicate. Some instruction sets do not include payment or other monetization related instructions.

At step 608, once payment is confirmed at step 607, system processor 508 communicates the satisfaction score to a third party interested in the satisfaction score. As discussed above, the interested third party may be a creator of the media content presented to the user. Alternatively, the interested third party may be a manufacturer, distributor, or retailer of a product advertised in marketing media content presented to the user.

Any currently known or later developed method of communicating the marketing metrics data may be used. For example, the marketing metrics data may be a file copied to a third party's computing system or network, such as over the internet or other distributed data network. In some examples, the marketing metrics data is communicated visually in an internet browser as a report, graph, or other display of data.

### Instructions Set 2

With reference to FIG. 10, the reader can see a variation of computer executable system instructions, system instructions 700. In system instructions 700, system processor 508 prompts a user to exhibit a therapeutic smile prior presenting media content to the user. Steps 701-706 and 708 in system instructions 700 are substantially like steps 601-606 and 608 discussed above. Unique steps 709 and 710 are discussed below.

A unique approach involved with system instructions 700 is prompting a user to exhibit a therapeutic smile at step 709 prior presenting media content to the user at step 701. Further, system instructions 700 uniquely includes at step 710 restricting presentation of media content until system processor 508 detects that user has exhibited a therapeutic smile.

In some examples, the media content includes entertainment content and marketing content and the user is prompted to smile therapeutically prior to presenting the marketing content, but not prior to presenting the entertainment content. In other examples, the user is prompted to smile therapeutically prior to presenting the entertainment content, but not prior to presenting the marketing content. In still further examples, the user is prompted to smile therapeutically prior to presenting both the marketing content and the entertainment content.

Prompting a user to exhibit a therapeutic smile prior to presenting media content at steps 709, 710, and 701 has been observed to have multiple functions and benefits. For instance, conditioning presentation of the media content on the user smiling therapeutically has been observed to effectively motivate the user to smile therapeutically. In this context, the media content serves as a reward.

Further, prompting the user to first smile places the user in a more receptive state of mind. Receptivity can enhance the media experience, help the user comprehend or retain information presented in the media content, and/or perceive the media content more favorably.

For example, a student prompted to smile therapeutically prior to being presented educational media content may have more enthusiasm for the lesson presented; comprehend and retain the lesson material more effectively; and have a generally positive impression of the lesson material and learning involved. In another example, a consumer prompted to smile therapeutically prior to being presented an advertisement may be more interested in the advertisement content; remember the advertisement more effectively when making purchasing decisions; and/or view the product advertised more favorably.

Detecting a therapeutic smile at step 710 may utilize the same instructions involved with steps 602-605 and 702-705; namely, receiving facial expression parameter data as described in instruction 702; receiving current facial expression data as provided in instruction 703; comparing the facial expression data as described in instruction 704; and analyzing the facial expression data as provided in step 705.

### Instructions Set 3

With reference to FIG. 11, the reader can see a variation of computer executable system instructions, system instructions 800. In system instructions 800, system processor 508 presents new media content after evaluating a user's facial expressions exhibited while observing the initial media content. Steps 801-805 in system instructions 800 are substantially like steps 601-605 discussed above. Unique steps 811-813 are discussed below.

System instructions 800 shown in FIG. 11 determines whether to present related or unrelated media content to the user based on how the user responds to the initial media content presented at step 801. In particular, subsequently presenting related or unrelated media content is based on a comparative assessment at steps 804, 805, and 811 of the user's current facial expression data received at step 803 and target facial expression criteria established at step 802. The current facial expression data received at step 803 corresponds to facial expressions exhibited while the user was consuming the initial media content presented at step 801.

For example, if a country music song is delivered initially and the media metrics data indicates at step 811 that a user does not respond favorably to it, system 500 may next deliver a blues song to the user at step 813. Here, blues music is defined to be unrelated to country music. In the same example, if the media metrics data indicates that the user responded favorably to the country music song at step 811, system 500 may next deliver another country song or perhaps another country song by the same artist to the user at step 812.

In another example utilizing system instructions 800 shown in FIG. 11, the media content presented at step 801 includes initial marketing content advertising a given product. If the media metrics data indicates at step 811 that a user does not respond favorably to the initial marketing content, system 500 next delivers subsequent, unrelated marketing content that differs from the initial marketing content at step 813. For example, the subsequent marketing content may be of a different style (such as inspiring rather than funny), but advertising the same given product. Additionally or alternatively, the subsequent marketing content may advertise a different product.

If the media metrics data indicates at step 811 that a user responds favorably to the initial marketing content, system 500 delivers subsequent media content related to the initial media content at step 812. The subsequent marketing content may advertise the same given product advertised in the initial media content. Additionally or alternatively, the subsequent marketing content may advertise a product like the given product advertised in the initial media content, such as a similar product by another brand and/or another product by the same brand as the given product advertised in the initial media content.

In some examples, the initial media content that system 500 presents to a user at step 801 includes initial marketing content targeting a specified marketing demographic. For example, the marketing content may target senior citizens or homeowners. Related marketing content subsequently presented to users at step 812 may market different products, but still target senior citizens or homeowners. Unrelated marketing content may target different demographics, such as teenagers or people renting apartments.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A media content metrics system comprising:
a facial expression detection device configured to acquire facial expression data of a person;
a system processor in data communication with the facial expression detection device and configured to execute stored computer executable system instructions; and
a media device controllably coupled to the system processor and configured to present media content to the person by playing media data;
wherein the computer executable system instructions include the steps of:
receiving facial expression parameter data establishing target facial expression criteria defining a smile;
receiving current facial expression data corresponding to the facial expression of the person from the facial expression detection device when the media content is presented to the person;
comparing the current facial expression data to the target facial expression criteria of the facial expression parameter data; and
establishing media metrics data for the media content based on the comparison of the current facial expression data to the target facial expression criteria.

2. The system of claim 1, wherein the media content includes entertainment content and marketing content.

3. The system of claim 4, wherein the computer executable system instructions further comprise:
prompting the person to exhibit a smile with therapeutic benefits prior to presenting the marketing content; and
restricting the media device from presenting the marketing content until the system processor identifies that the current facial expression data satisfies the target facial expression criteria.

4. The system of claim 1, wherein the computer executable system instructions further comprise identifying whether the current facial expression data satisfies the target facial expression criteria.

5. The system of claim 4, wherein the media metrics data includes how long the current expression data satisfied the target facial expression criteria.

6. The system of claim 4, wherein the media metrics data includes how quickly the current expression data fails to satisfy the target facial expression criteria.

7. The system of claim 1, wherein the media metrics data includes a satisfaction score corresponding to how well the current expression data satisfied the target facial expression criteria.

8. The system of claim 7, wherein the computer executable system instructions further comprise communicating the satisfaction score to a third party interested in the satisfaction score.

9. The system of claim 8, wherein the third party is a content creator that created the media content.

10. The system of claim 8, wherein:
the media content includes entertainment content and marketing content; and
the third party is a manufacturer, distributor, or retailer of a product advertised in the marketing content.

11. The system of claim 7, wherein the computer executable system instructions further comprise waiting to communicate the satisfaction score to the third party until a payment record indicates that the third party has paid a predetermined fee to receive the satisfaction score.

12. The system of claim 1, wherein the target facial expression criteria includes:
the zygomatic major muscles of the person contracting to a selected extent; and
the orbicularis oculi muscles of the person contracting to a selected extent.

13. The system of claim 1, wherein the facial expression detection device includes a camera.

14. The system of claim 1, wherein the facial expression detection device is incorporated into a handheld computing device.

15. The system of claim 1, wherein the facial expression detection device is incorporated into a watch.

16. The system of claim 1, wherein:
the media content presented to the user defines initial media content; and
the computer executable system instructions further comprise selecting subsequent media content to present to the person based on the comparison of the current facial expression data to the target facial expression criteria when presenting the initial media content.

17. The system of claim 16, wherein the computer executable system instructions further comprise selecting subsequent media content related to the initial media content when the current facial expression data satisfies the target facial expression criteria when presenting the initial media content.

18. The system of claim 17, wherein:
the initial media content includes initial marketing content advertising a given product; and
the subsequent media content related to the initial media content includes subsequent marketing content advertising the given product advertised in the initial media content.

19. The system of claim 17, wherein:
the initial media content includes initial marketing content targeting a specified marketing demographic; and
the subsequent media content related to the initial media content includes subsequent marketing content targeting the specified marketing demographic targeted in the initial marketing content.

20. The system of claim 16, wherein the computer executable system instructions further comprise selecting subsequent media content unrelated to the initial media content when the current facial expression data fails to satisfy the target facial expression criteria when presenting the initial media content.
